(19) United States
(12) Patent Application Publication
(10) Pub. No.: US 2023/0032239 A1
(43) Pub. Date: Feb. 2, 2023

(54) A METHOD OF TREATMENT IN PREDISPOSED SUBJECTS FOR LMNA-RELATED DILATED CARDIOMYOPATHY

(71) Applicant: The Board of Trustees of the Leland Stanford Junior University, Stanford, CA (US)

(72) Inventors: Vittavat Termglinchan, Palo Alto, CA (US); Ioannis Karakikes, Palo Alto, CA (US); Joseph C. Wu, Stanford, CA (US); Jaecheol Lee, Stanford, CA (US); Sebastian Diecke, Berlin (DE)

(21) Appl. No.: 17/791,110

(22) PCT Filed: Jan. 15, 2020
(86) PCT No.: PCT/US2020/013683

§ 371 Date: Jul. 6, 2022

(51) Int. Cl. A61K 31/4709; A61K 9/00

(52) U.S. Cl. CPC A61K 31/4709; A61K 9/0019; A61K 9/0056

(57) ABSTRACT

The present invention includes methods for treating and/or preventing dilated cardiomyopathy due to an LMNA mutation, comprised of administering to a subject at-risk a therapeutically effective amount of an inhibitor of PDGF signaling.

### STATEMENT OF FEDERALLY FUNDED RESEARCH

This invention was made with Government support under contract HL113006 awarded by the National Institutes of Health. The Government has certain rights in the invention.

### TECHNICAL FIELD

This disclosure pertains to methods of prevention and treatment of LMNA-mutated dilated cardiomyopathy in predisposed subjects in which an effective dose of crenolanib is administered to a subject at risk for LMNA-related dilated cardiomyopathy.

### BACKGROUND

Diagnosis of dilated cardiomyopathy (DCM) is principally characterized by left ventricular enlargement and/or a reduction in systolic function, more precisely described as a reduction in left ventricular ejection fraction (LVEF) less than <40% or fractional shortening less than 25% (Hershberger & Morales, 2016; Richardson et al., 1996; WHO/ISFC, 1980). In many cases for individuals with DCM, no etiology can be determined, therefore the cardiomyopathy is deemed idiopathic. In such a case, clinicians should suspect that a pathogenic variant of LMNA gene may be the underlying cause for DCM. Such cases account for approximately 5-10% of cases in humans (Brayson & Shanahan, 2017; Hershberger & Morales, 2016). Unlike other cases of familial DCM, often the first sign of LMNA-related DCM is sudden cardiac arrest leading to death, owing to prevalence of arrhythmias, e.g. ventricular tachycardia, ventricular fibrillation.

LMNA is a gene that encodes the intermediate filament proteins, lamin A and C, which localize between the nuclear membrane and the chromatin. The two protein isoforms of the LMNA gene are generated through alternative splicing of the pre-mRNA and lead to two unique proteins that even have differential post translational modifications (Goidescu, 2013; Lin & Worman, 1993). Lamin A/C plays a key role in maintaining nuclear shape and structure through contribution to the nuclear lamina. These proteins also affect the position and function of nuclear pores, regulation of translation and transcription, and chromatin organization (Goidescu, 2013). Mutations in the LMNA gene lead to disruption of cellular functions, which can result in a milieu of diseases referred to as laminopathies (Brayson & Shanahan, 2017; Lu, Muchir, Nagy, & Worman, 2011). Laminopathies all share a degree of nuclear fragility, altered nuclear architecture, impaired nuclear signaling and transcriptional activation through alterations in adaptive or protective mechanisms. LMNA is one of few established genes that has a clear genotype to clinical phenotype relationship, which has proven to be associated with conduction defects, malignant ventricular arrhythmias, and supraventricular arrhythmias preceding the development of left ventricular dilation and heart failure (Hershberger, Morales, & Siegfried, 2010).

Components of the platelet-derived growth factor (PDGF) signaling pathway are upregulated during the early phases of cardiomyocyte differentiation, but become downregulated in fully differentiated cardiomyocytes (Lee et al., 2019). Previous studies have shown that expression and activation of PDGFRβ dramatically increases in response to pressure overload-induced stress as is seen in hypertension, which has been associated with other clinically used PDGFR inhibitors (Chintalgattu et al., 2010). They further demonstrated that cardiomyocyte PDGFRβ knockout mice resulted in cardiac dysfunction, heart failure, and a marked defect in stress-induced cardiac angiogenesis, concluding that PDGFRβ is an essential regulator of paracrine angiogenic potential of cardiomyocytes (Chintalgattu et al., 2010). This would suggest that, under normal physiological conditions, the PDGF signaling pathway in adult cardiomyocytes is present but not active. Hyperactivation of this pathway can play a significant role in cardiac dysfunction, which can ultimately lead to heart failure (Lee et al., 2019).

Thus, there is a need for agents that can be used for the prevention and treatment of LMNA-related dilated cardiomyopathy in high-risk patients.

### SUMMARY

Methods are provided for treating dilated cardiomyopathy due to an LMNA mutation in a patient, the methods comprising administering to the patient an effective dose of an inhibitor of PDGF signaling. In some embodiments the inhibitor is type I mutant-specific inhibitor that preferentially binds to phosphorylated active kinases. In some embodiment the inhibitor is crenolanib (1-[2-[5-[(3-Methyl-3-oxetanyl) methoxy]-1H-benzimidazol-1-yl]- 8-quinolinyl]-monobenzenesulfonate) or a salt thereof. In some embodiments the effective dose reduces the progression of dilated cardiomyopathy. In some embodiments the effective dose prevents the further progression of dilated cardiomyopathy in an individual. In some embodiments the effective dose prevents the development of dilated cardiomyopathy in a susceptible individual. Prior to treatment, the individual may be diagnosed as having a genetic defect in LMNA associated with a predisposition to development of dilated cardiomyopathy. The genetic defect may be hereditary.

It is shown herein that LMNA-mediated hyperactivation of PDGFRβ signaling pathways in cardiomyocytes of an individual lead to changes in gene and protein expression that cause a proarrhythmic phenotype. In some embodiments, the methods of treatment described herein prevent or reduce LMNA-mediated hyperactivation of PDGFRβ in cardiomyocytes. In some embodiments, the methods of treatment described herein reduce the level of phosphorylation of CAMK2D and RYR2 in cardiomyocytes of the individual. In some embodiments the methods of treatment described herein reduce the pro-arrhythmic phenotype of cardiomyocytes carrying an LMNA mutation.

In an aspect, the effective amount of crenolanib is from about 50 mg to 500 mg per day, 100 to 450 mg per day, 200 to 400 mg per day, 300 to 500 mg per day, 350 to 500 mg per day, or 400 to 500 mg per day. In another aspect, the effective amount of crenolanib is administered at least one of continuously, intermittently, systemically, or locally. In yet another aspect, the effective amount of crenolanib is administered orally, intravenously, or intraperitoneally. In another aspect, the effective amount of crenolanib is administered up to three times a day for as long as the subject is at risk for development of LMNA-related dilated cardiomyopathy. In another aspect, the crenolanib is crenolanib besylate, crenolanib phosphate, crenolanib lactate, crenolanib hydrochloride, crenolanib citrate, crenolanib acetate, crenolanib toluene sulphonate, or crenolanib succinate.

### BRIEF DESCRIPTION OF THE DRAWINGS

For a more complete understanding of the features and advantages of the present invention, reference is now made to the detailed description of the invention along with the accompanying figures and in which:

FIGS. 1A to 1D show the ability of the present invention to inhibit the pro-arrhythmic phenotype in LMNA-related dilated cardiomyopathy patient samples. Patient-derived induced pluripotent stem cell-derived cardiomyocytes (iPSC-CMs) with an LMNA mutation were treated with crenolanib (100 nM) for 24 h to determine the effects on PDGFR signaling and pro-arrhythmic phenotype. FIG. 1A: Quantification of Ca2+ transients was used to determine the percentage of cells that exhibit an arrhythmic phenotype for mutant iPSC-CMs treated with DMSO (control) or PDGFR inhibitors crenolanib (CB; 100 nM) or sunitinib (SB; 500 nM). Treatment with crenolanib decreased the percentage of cells with a pro-arrhythmic phenotype. FIG. 1B: Immunoblot analysis of phosphorylated CAMK2D, which is involved in abnormal Ca2+ signaling and the development of arrhythmia in this model system, showed that treatment with crenolanib reduced the levels of pCAMK2D. FIG. 1C and D: Gene ontology (GO) analysis identified a set of genes related with muscle contraction and regulation of cardiac conduction that was downregulated in crenolanib-treated samples, indicating that crenolanib may have an effect on systolic dysfunction as well as arrhythmia associated with dilated cardiomyopathy.

FIGS. 2A-2J shows the ability of the present invention to inhibit the PDGFRβ pathway, reducing the arrhythmic phenotype of LMNA mutant iPSC-CMs. Arrhythmic phenotype in mutant iPSC-CMs is dependent on the activation of the PDGFRB pathway. FIG. 2A, qPCR analysis of PDGFRB expression levels in mutant iPSC-CMs (WT/MUT) treated with scramble or PDGFRB siRNAs. The cells were treated with siRNAs for 48 h. Data are mean ± s.e.m.; a two-tailed Student's t-test was used to calculate P values; n = 3; the value above the line indicates significance. FIG. 2B, Representative Ca2+ transients of mutant iPSC-CMs (lll-17 WT/MUT) treated with scramble siRNA or PDGFRB siRNA. FIG. 2C, Quantification of the number of cells that exhibited arrhythmic waveforms in b. FIG. 2D, Representative Ca2+ transients of mutant iPSC-CMs treated with PDGRB inhibitors, crenolanib (100 nM) and sunitinib (500 nM), for 24 h. All traces were recorded for 20 s. FIG. 2E, Quantification of mutant iPSC-CMs (lll-17, lll-15 and lll-3) that exhibited arrhythmic waveforms with or without the treatment of PDGRB inhibitors, crenolanib (100 nM) and sunitinib (500 nM), for 24 h. FIG. 2F, Representative Ca²⁺transients of mutant iPSC-CMs (lll-17 WT/MUT) treated with PDGFRB inhibitors. FIG. 2G, Immunoblot analysis of pRYR2 and RYR2 protein levels with treatment of DMSO, crenolanib or sunitinib. The data were repeated twice independently with similar results. FIG. 2H, Immunoblot analysis of PDGFRB, tubulin, pCAMK2D and CAMK2D protein levels in control iPSC-CMs expressing empty and PDGFRB constructs. The signal intensity of the PDGFRB (left) and p-CAMK2D (right) is shown. The experiments were repeated twice independently with similar results. FIG. 2I, Representative Ca²⁺ transients of iPSC-CMs expressing empty and PDGFRB constructs. FIG. 2J, Quantification of arrhythmic waveforms of iPSC-CMs in I.

FIGS. 3A-H. Gene-expression profile of PDGFRB inhibition in LMNA-mutant iPSC-CMs. FIG. 3A, GO analysis of downregulated genes (n=352) in LMNA-mutant iPSC-CMs treated with PDGFRB inhibitors, crenolanib (100 nM) and sunitinib (500 nM), for 24 h. FIG. 3B, Heat map of the expression profile of the gene set related to the GO function of ion transport. The FDR-adjusted P values were obtained using the GO enrichment analysis tool. FIG. 3C, Hierarchical clustering of AmpliSeq RNA-seq data using one-way ANOVA (p=0.05; n=230). Two different siRNAs against PDGFRB and a scramble siRNA were used in LMNA-mutant iPSC-CMs (lll-15 WT/MUT). FIG. 3D, FIG. 3E, Heat map of expression profile of gene (n = 25) sets related with the GO function of cardiac muscle contraction (d) and actin-mediated cell contraction (e). The FDR-adjusted P values were obtained using the GO enrichment analysis tool. FIG. 3F, No significant changes in abnormal nuclear structures of mutant iPSC-CMs by inhibition of PDGFRB were found. Representative images of mutant iPSC-CMs treated with PDGFRB inhibitors, crenolanib (100 nM) and sunitinib (500 nM), for 24 h. iPSC-CMs were stained with specific antibodies against LMNB1 (green). Blue, DAPI. Scale bars, 10 µm . The experiments were repeated three times independently with similar results. FIG. 3F, Quantification of cells showing abnormal nuclear structures in mutant iPSC-CMs treated with PDGFRB inhibitors. The images were recorded from three differentiation batches. n = 90 (DMSO), n = 69 (crenolanib), n = 79 (sunitinib). Data are mean ± s.e.m.; statistical significance was analyzed using one-way ANOVA; values above the lines indicate significance. FIG. 3H, Immunoblot analysis of lamin A/C and GAPDH protein levels in mutant iPSC-CMs treated with PDGFRB inhibitors. CB, crenolanib; SB, sunitinib. The experiments were repeated twice independently with similar results.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity but include the general class of which a specific example may be used for illustration. The terminology herein is used to describe specific embodiments of the invention, but their usage does not delimit the invention, except as outlined in the claims.

### Definitions

As used herein, the term “subject” or “patient” are used interchangeably to refer to an animal, such as a mammal or a human, who has been the object of treatment, observation, or experiment.

As used herein, the terms “prevent” and “prevention” refer to administering crenolanib or a pharmaceutical salt thereof to a patient or subject, prior or during to the onset of a disease, disorder, condition or symptom thereof, so as to prevent, suppress, inhibit or reduce, either temporarily or permanently, a subject’s risk of developing LMNA-related dilated cardiomyopathy or delaying the onset thereof.

As used herein, the terms “predisposed” “subject at risk” refer to a subject or patient that has one or more risk factors for a disease, for example, genetic or other factors (such as an LMNA mutation) that can cause the subject to develop LMNA-related dilated cardiomyopathy. For example, a subject is predisposed to LMNA-related dilated cardiomyopathy if the one or more factors indicate the possible development of LMNA-related dilated cardiomyopathy, but the subject does not yet experience or exhibit symptoms of the disease.

As used herein, the term “in need of prevention” refers to a judgment made by a physician or other caregiver that a subject or patient requires or will benefit from preventative care. This judgment is made based on a variety of factors that are in the realm of a physician's or caregiver’s expertise.

As used herein, the terms “treat”, “treating”, and “treatment” refer to the administration of one or more active ingredients, compounds, salts, or compositions that prevent, reduce, or delay the onset of the symptoms or complications of LMNA-related dilated cardiomyopathy. “Treating” further refers to any indicia of success in the treatment or amelioration or prevention of the disease, condition, or disorder, including any objective or subjective parameter such as abatement; diminishing of symptoms or making the disease condition more tolerable to the patient; slowing in the rate of degeneration or decline; or making the disease less debilitating. The treatment or amelioration of symptoms can be based on objective or subjective parameters; including the results of an examination by a physician. Accordingly, the term “treating” includes the administration of the compounds, salts, or agents of the disclosure to prevent or delay, to alleviate, or to arrest or inhibit development of the symptoms or conditions associated with LMNA-related dilated cardiomyopathy and heart failure.

As used herein, the terms “heart failure” refers to a chronic, progressive condition in which the heart muscle is unable to pump enough blood to meet the body's needs for blood and oxygen. LMNA-related dilated cardiomyopathy can occur in different types of animals and humans.

As used herein, the term “LMNA-related dilated cardiomyopathy” refers to a heart condition in which a subject is affected by left ventricular enlargement and/or reduced systolic function preceded or accompanied by significant conduction system disease and/or arrhythmias due to pathological variant in the LMNA gene.

LMNA mutations. DCM associated with mutations in LMNA (LMNA-related DCM) is an autosomal dominant disorder caused by mutations in the gene that encodes the lamin A/C proteins that constitute the major component of the nuclear envelope. LMNA-related DCM accounts for 5-10% of cases of DCM and has an age-related penetrance with a typical onset between the ages of 30 and 40. In contrast to most other forms of familial DCM, sudden cardiac death may be the first manifestation of LMNA-related DCM even in the absence of systolic dysfunction, owing to malignant arrhythmias such as ventricular tachycardia and fibrillation. See, for example, Carmosino, M. et al. Biol. Cell 106, 346-358 (2014); Fatkin, D. et al. N. Engl. J. Med. 341, 1715-1724 (1999); and Krohne, G. & Benavente, R. The nuclear lamins. Exp. Cell Res. 162, 1-10, each herein specifically incorporated by reference. A predisposing mutation causes a change in lamin sequence or expression that leads to malignant arrhythmias and development of DCM. Conduction system disease can be detected by a 12-lead electrocardiogram (ECG); arrhythmias can be detected by an ECG, 24-hour rhythm recording, or event monitor. Left ventricular enlargement can be diagnosed with cardiac imaging; reduced systolic function is assessed by two-dimensional echocardiography, angiography, radioisotope scanning, or magnetic resonance imaging.

Specific sequence of LMNA associated DCM include, without limitation: R60G; L85R; Asn195Lys; Glu203Gly; Arg571Ser; K117fs; N195K; H222P; G608G; M371K; ΔK32; L530P; E82K; R26G; K32del; R249Q; R249Q; Y267C; R453W; T528R; R377H; ARG60GLY; LEU85ARG; ASN195LYS; GLU203GLY; ARG571SER; GLU161LYS; 1-BP INS, 28A; ALA57PRO; SER573LEU; LEU59ARG; ARG541GLY; etc.

LMNA sequence analysis can be used to identify pathogenic variants in most individuals with LMNA-related DCM. Various methods known in the art can be used for analysis of the genotype of these genes. Traditional methods for detecting mutations involved screening by direct DNA sequencing of the tumor tissue. Sanger sequencing technology is available in most molecular diagnostic laboratories, and it has the singular advantage of detecting alterations across a gene, including novel variants. Recent methodologies have focused on targeted screening of mutations to achieve more rapid, robust, and sensitive tests. Molecular diagnostic laboratories currently use a variety of methods, including amplification refractory mutation system, pyrosequencing, smart amplification process, high-resolution melting analysis, and restriction fragment length polymorphism, to name a few. These methods all distinguish between mutant and wild-type DNA within the region of interest. In contrast to direct sequencing, the limit of detection for targeted analysis is ~1-5% mutant DNA in the background of normal DNA. Formalin-fixed, paraffin-embedded (FFPE) tissue can be used to test for mutations. Alternate sample types such as fine needle aspirates and pleural effusions are currently being evaluated as viable options to enable quicker, easier diagnosis.

PDGF inhibitor. These inhibitors act selectively to inhibit PDGF signaling. The PDGF family is a product of four gene products and consists of five dimeric isoforms: PDGF-AA, PDGF-BB, PDGF-CC, PDGF-DD, and the PDGF-AB heterodimer. These growth factors mediate their effects by binding to and activating their receptor protein-tyrosine kinases, which are encoded by two genes: PDGFRA and PDGFRB. The functional receptors consist of the PDGFRα/α and PDGFRβ/β homodimers and the PDGFRα/β heterodimer. The PDGF receptors contain an extracellular domain that is made up of five immunoglobulin-like domains, a transmembrane segment, a juxtamembrane segment, a protein-tyrosine kinase domain that contains an insert of about 100 amino acid residues, and a carboxyterminal tail.

Type I protein kinase inhibitors interact with the active enzyme form with DFG-D of the proximal activation segment directed inward toward the active site (DFG-Dᵢₙ). In contrast, type II inhibitors bind to their target with the DFG-D pointing away from the active site (DFG-Dₒᵤₜ). Inhibitors of interest are known and used in the art and may include, without limitation, Crenolanib; Imatinib; Sunitinib; Sorafenib; Pazopanib; Nilotinib; Cediranib; Motesanib; Axitinib; Linifenib; Dasatinib; Quizartinib; Ponatinib. In some embodiments the inhibitor is Crenolanib. In some embodiments the inhibitor is Sunitinib.

Crenolanib (4-Piperidinamine, 1-[2-[5-[(3-methyl-3-oxetanyl) methoxy]-1H-benzimidazol-1-yl]-8-quinolinylJ) and its pharmaceutically acceptable salts, include without limitation: Crenolanib Besylate, Crenolanib Phosphate, Crenolanib Lactate, Crenolanib Hydrochloride, Crenolanib Citrate, Crenolanib Acetate, Crenolanib Toluenesulphonate and Crenolanib Succinate, but may also be made available free of salts. Preparation of the compounds of the present invention. General synthetic methods for preparing the compounds of Formula I are provided in, e.g., U.S. Pat. No. 5,990,146 (issued Nov. 23, 1999) (Warner-Lambert Co.) and PCT published application numbers WO 99/16755 (published Apr. 8, 1999) (Merck & Co.) WO 01/40217 (published Jul. 7, 2001) (Pfizer, Inc.), U.S. Pat. Application Publication No. US 2005/0124599 (Pfizer, Inc.) and U.S. Pat. No. 7,183,414 (Pfizer, Inc.), relevant portions incorporated herein by reference. Crenolanib is an orally bioavailable, selective, and potent type I tyrosine kinase inhibitor (TKI) of class III receptor tyrosine kinases (RTKs). The compound has the ability to inhibit both PDGFRα and PDGFRβ. Crenolanib does not inhibit any other known RTKs (e.g., VEGFR or fibroblast growth factor receptor) at concentrations that are used clinically.

As used herein, the term "therapeutically effective amount" refers to an amount of crenolanib or a pharmaceutically acceptable salt thereof, administered to a subject as a single agent or in combination with another pharmaceutical agent(s), e.g., a chemotherapeutic agent, that in combination elicits the biological or medicinal response in a subject that is being sought by a researcher, veterinarian, medical doctor, or other clinician, which includes alleviation of the symptoms of the disease or disorder being treated. Methods for determining therapeutically effective doses for pharmaceutical compositions comprising a compound of the present invention are known in the art. Techniques and compositions for making useful dosage forms using the present invention are described in many references, including: P. O. Anderson, J. E. Knoben, and W. G. Troutman, Handbook of clinical drug data, 10th ed. New York; Toronto: McGraw-Hill Medical Pub. Division, 2002, pp. xvii, 1148 p (Anderson, Knoben, & Troutman, 2002); A. Goldstein, W. B. Pratt, and P. Taylor, Principles of drug action : the basis of pharmacology, 3rd ed. New York: Churchill Livingstone, 1990, pp. xiii, 836 p.(Goldstein, Pratt, & Taylor, 1990); B. G. Katzung, Basic & clinical pharmacology, 9th ed. (Lange medical book). New York: Lange Medical Books/McGraw Hill, 2004, pp. xiv, 1202 p.(Katzung, 2004); L. S. Goodman, J. G. Hardman, L. E. Limbird, and A. G. Gilman, Goodman and Gilman's the pharmacological basis of therapeutics, 10th ed. New York: McGraw-Hill, 2001, pp. xxvii, 2148 p.(Goodman, Hardman, Limbird, & Gilman, 2001); J. P. Remington and A. R. Gennaro, Remington : the science and practice of pharmacy, 20th ed. Baltimore, Md.: Lippincott Williams & Wilkins, 2000, pp. xv, 2077 p.(Remington & Gennaro, 2000); W. Martindale, J. E. F. Reynolds, and Royal Pharmaceutical Society of Great Britain. Council, The extra pharmacopoeia, 31st ed. London: Royal Pharmaceutical Society, 1996, pp. xxi, 2739 p.(Martindale, Reynolds, & Royal Pharmaceutical Society of Great Britain. Council, 1996); and G. M. Wilkes, Oncology Nursing Drug Handbook 2016, 20 ed. Sudbury: Jones & Bartlett Publishers, 2016, p. 1500 p.(Wilkes, 2016), relevant portions of each are incorporated herein by reference.

### Methods

The present invention is based, at least in part, on the discovery that PDGFRβ is abnormally expressed in LMNA-related dilated cardiomyopathy, and that the inhibition thereof results in decreased arrhythmic potential which may benefit patients at risk of developing this disease. The present invention comprises the use of PDGF inhibitors, e.g. crenolamib, etc. for prevention of LMNA-related dilated cardiomyopathy in high-risk patients.

In one embodiment, the present invention provides a method to prevent and/or treat dilated cardiomyopathy by inhibiting PDGFRβ signaling in a subject at risk of developing LMNA-related dilated cardiomyopathy. This comprises administering to a subject an effective dose of an inhibitor of PDGFβ signaling, which inhibitors include crenolamib and salts thereof.

In one aspect of this invention, the PDGF inhibitor is administered to a subject systemically, for example, orally, intravenously, subcutaneously, intramuscular, intradermal, or parenterally. The compound of the present invention can also be administered to a subject locally.

The PDGF inhibitor of the present invention may be formulated for slow-release or fast-release with the objective of maintaining contact of compounds of the present invention with targeted tissues for a desired range of time.

Compositions suitable for oral administration include solid forms, such as pills, tablets, caplets, capsules, granules, and powders, liquid forms, such as solutions, emulsions, and suspensions. Forms useful for parenteral administration include sterile solutions, emulsions, and suspensions.

The daily dosage of the PDGF inhibitor may be varied over a wide range from 50 to 500 mg per adult human per day. For oral administration, the compositions are preferably provided in the form of tablets containing 20 to 100 milligrams. The PDGF inhibitor may be administered on a regimen up to three times or more per day. Optimal doses to be administered may be determined by those skilled in the art and will vary with the compound of the present invention used, the mode of administration, the time of administration, the strength of the preparation, and the details of the disease condition. Factors associated with patient characteristics, such as age, weight, and diet will call for dosage adjustments.

Pharmaceutically acceptable salts such as hydrochloride, phosphate, and lactate are prepared in a manner similar to the benzenesulfonate salt and are well known to those of moderate skill in the art. The following representative compounds as PDGF inhibitors are for exemplary purposes only and are in no way meant to limit the invention, including crenolanib as crenolanib besylate, crenolanib phosphate, crenolanib lactate, crenolanib hydrochloride, crenolanib citrate, crenolanib acetate, crenolanib toluenesulphonate, and crenolanib succinate.

As used herein, patients considered at risk for developing LMNA-related dilated cardiomyopathy have a mutation in LMNA or are related to patients having mutations in LMNA. Genetic screening may be performed prior to treatment to identify individuals as risk. The 2009 Heart Failure Society of America (HFSA) guidelines note that the finding of a specific mutation does not generally govern therapy, although certain clinical characteristics associated with some genes may influence screening, education, and counseling of family members, and the threshold for primary prevention or pre-symptomatic therapy (Hershberger et al., 2009).

Conventional pharmacological treatment for patients with LMNA-related dilated cardiomyopathy may comprise treatment with ACE inhibitors, beta blockers, and/or anti-aldosterone agents, and some experts recommend anticoagulation (Hershberger & Morales, 2016). Furthermore, the 2009 HFSA guidelines recommends medical or device therapies recommended based on cardiac phenotype. Cardiac transplantation or other advanced therapies may be considered for refractory disease in persons receiving comprehensive care from cardiovascular disease experts (Hershberger & Morales, 2016). In addition, in patients with dilated cardiomyopathy (DCM) and significant arrhythmia or known risk of arrhythmia, an implantable cardioverter-defibrillator may be considered before the LV ejection fraction (LVEF) falls to ≤35 percent (the usual LVEF threshold for prophylactic implantable cardioverter-defibrillator placement). Specifically, an implantable cardioverter-defibrillator may be considered in patients with DCM with EF >35 percent with family history of sudden cardiac death OR with LMNA mutation (associated with high risk of sudden death; (Hershberger et al., 2009)). Any of these therapies may be provided in combination with the methods of treatment disclosed herein.

### EXAMPLES

### Example 1. Prevention of Arrhythmia and Systolic Dysfunction in Subjects at Risk of LMNA-Related Dilated Cardiomyopathy

Lamin A/C proteins are key components of heterochromatin conformation and the gene-silencing machinery and are expressed in a cell-type-specific manner (Mattout, Cabianca, & Gasser, 2015; Perovanovic et al., 2016; Solovei et al., 2013). (Lee et al., 2019)). The study performed by Lee et al. (2019) demonstrated that PDGFRB inhibitors can be repurposed for the treatment of dilated cardiomyopathy. The data presented herein elucidate how lamin A/C haploinsufficiency affects patient-derived iPSC-CMs and the development of arrhythmia. Furthermore, the inhibition of the PDGF pathway with inhibitors, including the present invention, ameliorates the arrhythmic phenotype of LMNA-mutant iPSC-CMs and downregulates genes associated with systolic dysfunction and heart failure, suggesting a novel therapeutic target for the treatment of LMNA-related DCM (Reproduced from (Lee et al., 2019)).

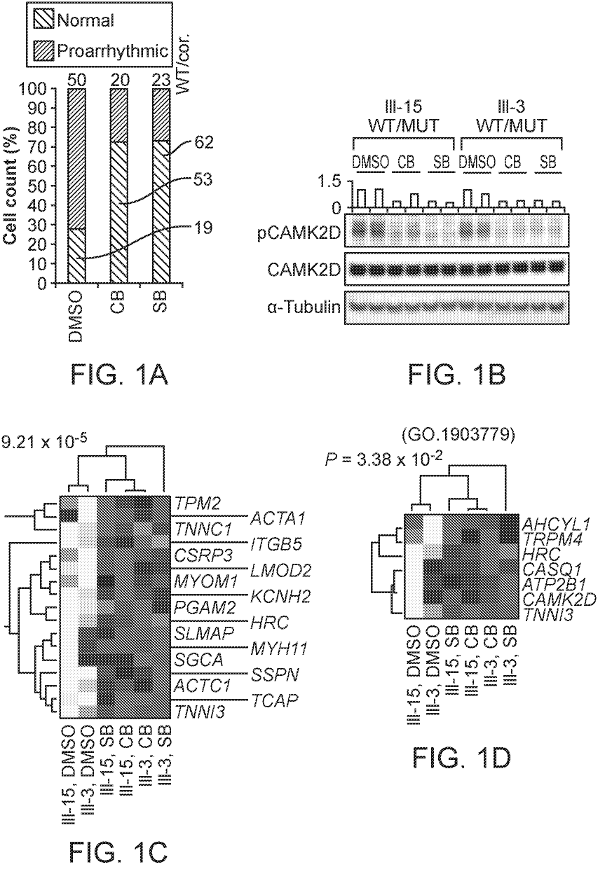

Multiple patient-derived iPSC lines were generated using nonintegrating reprogramming methods (Diecke et al., 2015; Kodo et al., 2016). Differentiation into cardiomyocytes (iPSC-CMs) was achieved using a chemically defined protocol (Lee et al., 2018). The LMNA-mutant iPSC-CMs (lll-3, III-9, III-15 and III-17) exhibited proarrhythmic activity in both atrial- and ventricular-like iPSC-CMs compared to healthy controls (FIGS. 1A and 1B). Taken together, these data demonstrate that iPSC-CMs derived from patients with lamin A/C haploinsufficiency recapitulate the disease phenotype associated with LMNA-related DCM in vitro.

A panel of isogenic lines was generated that differed only in this mutation using the iPSC line derived from patient III-3 (who carried one wild-type and one mutant allele (WT/MUT)) through TALEN mediated genome editing. Specifically, the LMNA mutation was corrected to the wild-type allele in the iPSCs (WT/cor-WT). The K117fs mutation was inserted in the wild-type allele (ins-MUT/MUT) and a knockout iPSC line generated by targeting the start codon (ATG site) of the wild-type allele (del-KOiMUT). Rhe K117fs mutation was introduced into in the healthy control iPSC line (patient IV-1, who carried two wild-type alleles (WT/WT)) to generate a heterozygous mutant iPSC line (WT/ins-MUT). We generated iPSC-CMs from the isogenic lines and observed that the targeted gene correction rescued the electrophysiological abnormalities in WT/cor-WT-derived iPSC-CMs compared to parental WT/MUT, genome edited ins-MUT/MUT and del-KO/MUT iPSC-CMs. The insertion of the K117fs mutation in the line derived from the healthy control individual (WT/ins-MUT) induced arrhythmias. Together, these data confirm that LMNA K117fs is a pathogenic mutation that causes LMNA-related DCM.

As homeostasis of Ca²⁺ is critical for excitation-contraction coupling in the heart, the intracellular Ca²⁺-handling properties of the patient-derived cells were measured. iPSC-CMs were seeded on glass coverslips to 5-7 days and loaded with the cell-permeable calcium-sensitive dye fura-2 AM for 20 min. After washing in buffer to allow de-esterification, coverslips were pointed on an inverted epifluorescence microscope. Cells were field-stimulated at 0.5 Hz with a pulse duration of 10 ms. Fura-2-AM-loaded cells were excited at both 340 and 380 nm, and the emission fluorescence signal was collected at 510 nm as previous described (Lam et al., 2013). Changes in fluorescence signal were measured using the NIS Elements AR software, which permits the recording of multiple cells in one view. Intracellular calcium changes were expressed as changes in the ratio R=F340/F380 and the calcium transient waves analyzed using a previously published method (Greensmith, 2014). Abnormal Ca2+ transients directly corresponded to arrhythmic phenotypes in this model system. Wildtype iPSC-CMs displayed a normal Ca2+ transient waveform, while mutant iPSC-CMs showed abnormal peaks corresponding to arrhythmia as measure by patch-clamp recordings (FIG. 1A). Treatment with the PDGFRβ inhibitors crenolanib (CB; 100 nM) or sunitinib (SB; 500nM) returned Ca2+ transient handling to normal functions (FIG. 1B). The percentage of measured cells displaying normal or arrhythmic phenotypes was recorded and represented as column graphs (FIG. 1C).

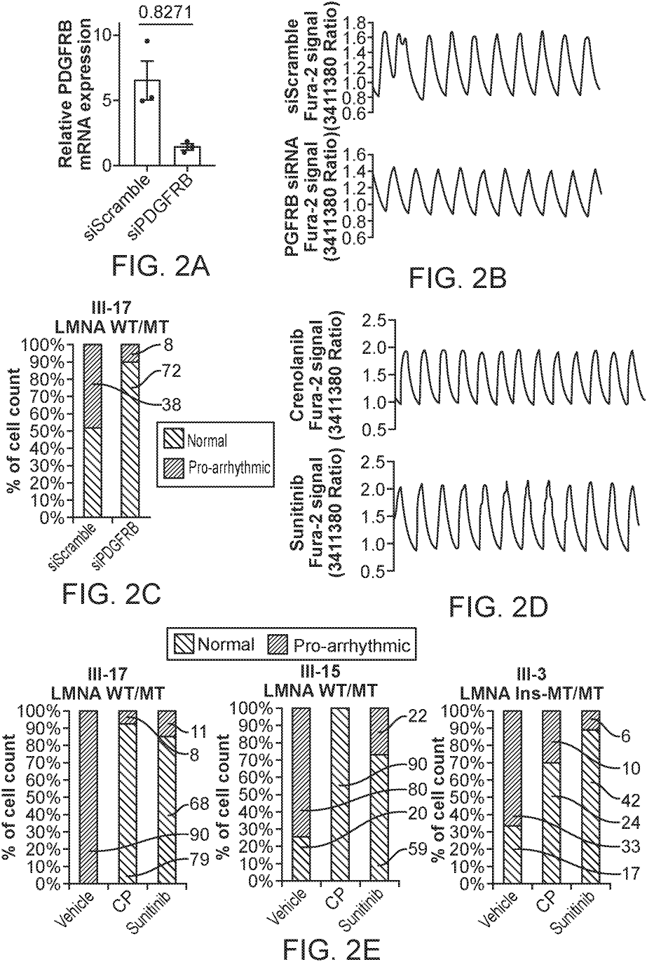

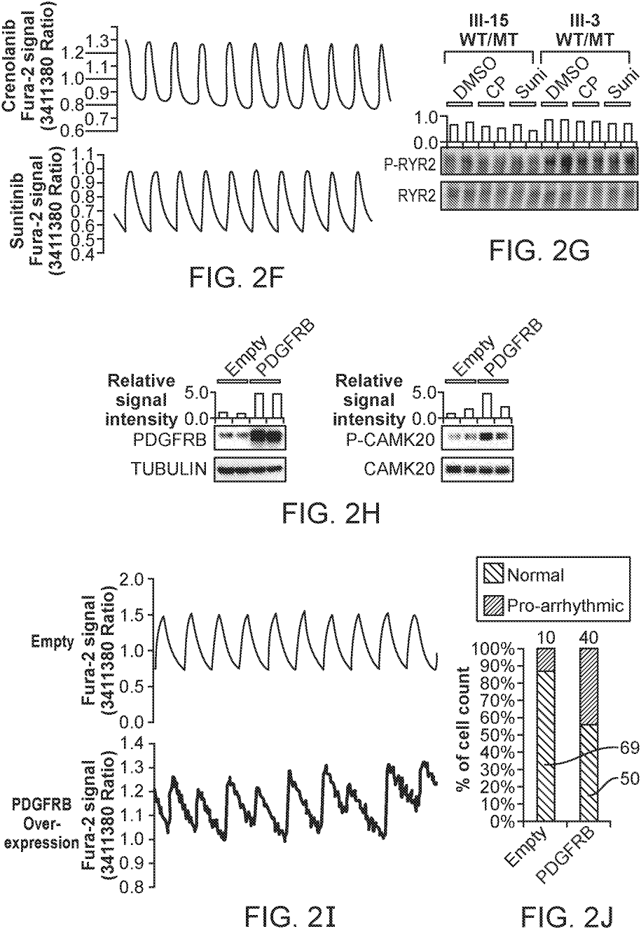

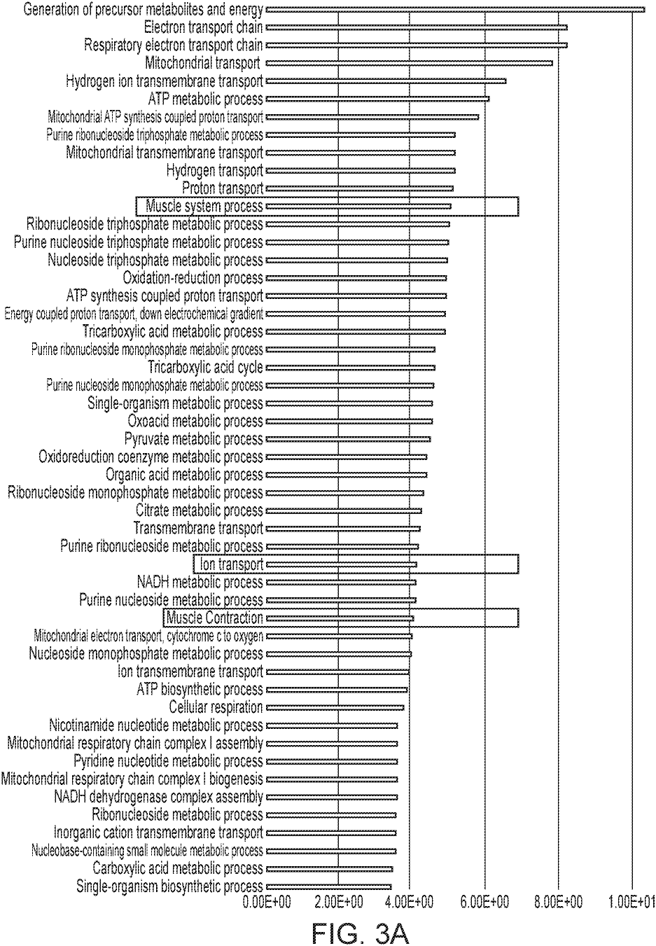

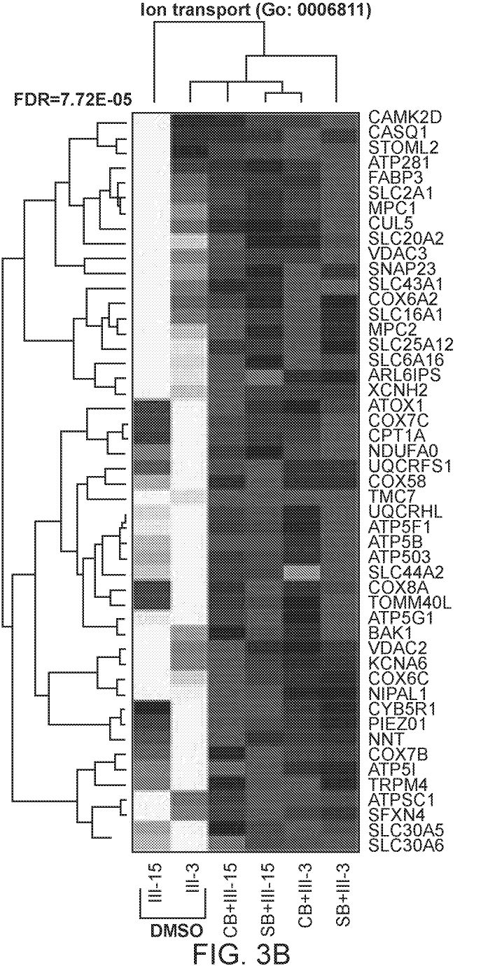

As phosphorylation of CAMK2D is involved in intracellular calcium signaling and may link PDGFR signaling to the arrhythmic phenotype, western blotting was used to determine the effect of PDGFR blockade on phosphorylation of CAMK2D (pCAMK2D). Proteins were resolved by SDS-PAGE and were transferred to 0.45-µm nitrocellulose membranes using a mini in NuPAGE transfer buffer. The membrane was then blocked and incubated with primary antibodies overnight at 4° C. Blots were incubated with the appropriate secondary antibodies for 1 h at room temperature and visualized using the ECL. Primary antibodies used were mouse anti-LMNA, rabbit anti-LMNA, CAMK2D, PDGFRB, RYR2, pRYR2 and HRP-conjugated a-tubulin. Cells treated with PDGFR inhibitors were more likely to display normal calcium signaling (FIG. 2A), and this corresponded to a decrease in the levels of pCAMK2D (FIG. 2B). This shows that the present invention is capable of reducing the levels of pCAMK2D and restoring a normal rhythm function to LMNA-mutated cardiomyocytes.

The PDGF pathway links to arrhythmic phenotype. To identify additional potential target genes that are closely associated with the disease phenotype, we compared the transcriptomes of K117fs mutant and control iPSC-CMs. By comparing the total RNA expression of control iPSC-CMs versus K117fs iPSC-CMs, we found that most of the differentially expressed genes were upregulated in K117fs iPSC-CMs (III-3, 84.87%; IV-1, 70.80%). A cross-analysis of differentially expressed genes based on two different genetic backgrounds (III-3 and IV-1) identified 257 genes for which the expression in K117fs iPSC-CMs significantly differed from that in isogenic control iPSC-CMs. As expected, 239 out of 257 genes (93%) were upregulated in K1 17fs iPSC-CMs compared to isogenic control iPSC-CMs. Gene ontology (GO) enrichment analysis revealed that the upregulated genes in K117fs iPSC-CMs were functionally enriched in terms associated with platelet-derived growth factor (PDGF) binding arylsulfatase activity, protein binding involved in cell-matrix adhesion and PDGF receptor binding.

The ARCHS4 kinase analysis also showed that the upregulated genes in K117fs iPSC-CMs were highly enriched in the PDGF pathway. PDGF signaling is initiated through the activation of two major receptors belonging to the PDGF receptor family, PDGFR-α (PDGFRA) and PDGF receptor-β (PDGFRB). During cardiomyocyte differentiation, PDGFRA and PDGFRB are highly upregulated in the early stages of differentiation but become downregulated after generating functional cardiomyocytes. In particular, expression of PDGFRB mRNA and PDGFRB protein is low in adult iPSC-CMs and normal heart tissues, but can be increased by stress conditions, which suggests that the PDGF signaling pathway is silenced in cardiomyocytes under physiological conditions.

A significant increase in PDGFRB mRNA and protein expression occurred in K117fs iPSC-CMs compared to control iPSC-CMs. In addition, a kinase array showed hyperactivation of PDGFRB in K117fs iPSC-CMs compared to isogenic control iPSC-CMs. Furthermore, the promoter region of the PDGFRB was more accessible in K117fs iPSC-CMs, as demonstrated by high enrichment of an active histone marker (H3K4me3) and open chromatin in the ATAC-seq analysis. Consistent with our observations in iPSC-CMs, heart tissue samples from both patients with LMNA-related DCM showed lower LMNA expression and higher PDGFRB expression when compared to healthy control tissues. Taken together, these data show that PDGFRB is epigenetically activated in K117fs iPSC-CMs.

The abnormal activation of PDGFRB was tested for a direct linkage to the arrhythmic phenotype that was observed in K117fs iPSC-CMs. Knockdown of PDGFRB expression in K117fs iPSCCMs by small interfering (si)RNA resulted in a reduced prevalence of abnormal Ca2+ transients (23.28%, n = 72) compared to the treatment with scramble siRNA control (100%, n = 75).

To test the effects of the abnormal activation of PDGFRB on the gene-expression profile of K117fs iPSC-CMs, we evaluated how treatment with crenolanib and sunitinib affected the transcriptome of K117fs iPSC-CMs. The PDGFRB inhibitors sunitinib and crenolanib (Selleckchem) were dissolved in DMSO. An equal concentration of solvent (DMSO) was used as the control. iPSC-CMs were treated with sunitinib or crenolanib for 48 h before the experiment.

Shown in FIG. 2, treatment with two specific PDGFRB inhibitors, crenolanib and sunitinib, ameliorated the arrhythmic phenotype of K117fs iPSC-CMs (crenolanib 27.39%, n = 73; sunitinib 27.05%, n = 85) compared to DMSO-treated cells (72.46%, n = 69). The phosphorylation of both CAMK2D and RYR2 was reduced after treatment of K117fs iPSC-CMs with crenolanib or sunitinib (III-15 and III-3). We also observed that the overexpression of PDGFRB resulted in upregulation of CAMK2D phosphorylation, inducing an arrhythmic phenotype in control iPSC-CMs (44.44%, n = 90). These data indicate that the abnormal activation of PDGFRB contributes to the arrhythmic phenotype observed in K117fs iPSC-CMs.

In order to determine the effect of PDGFRβ inhibition on the expression of genes associated with muscle contraction and regulation of cardiac conduction, reverse transcription and quantitative PCR were used, and gene ontology enrichment analysis was used. Total mRNA was isolated from iPSC-CMs. Subsequently, 1 µg of RNA was used to synthesize cDNA using the iScript. Then, 0.25 µl of the reaction was used to quantify gene expression by qPCR using TaqMan master mix. Expression values were normalized to the average expression of the housekeeping gene 18S. These studies showed that treatment of LMNA-mutated cardiomyocytes with PDGFRβ inhibitors downregulated a number of genes associated with muscle contraction (FIG. 2C) and regulation of cardia conduction (FIG. 2D).

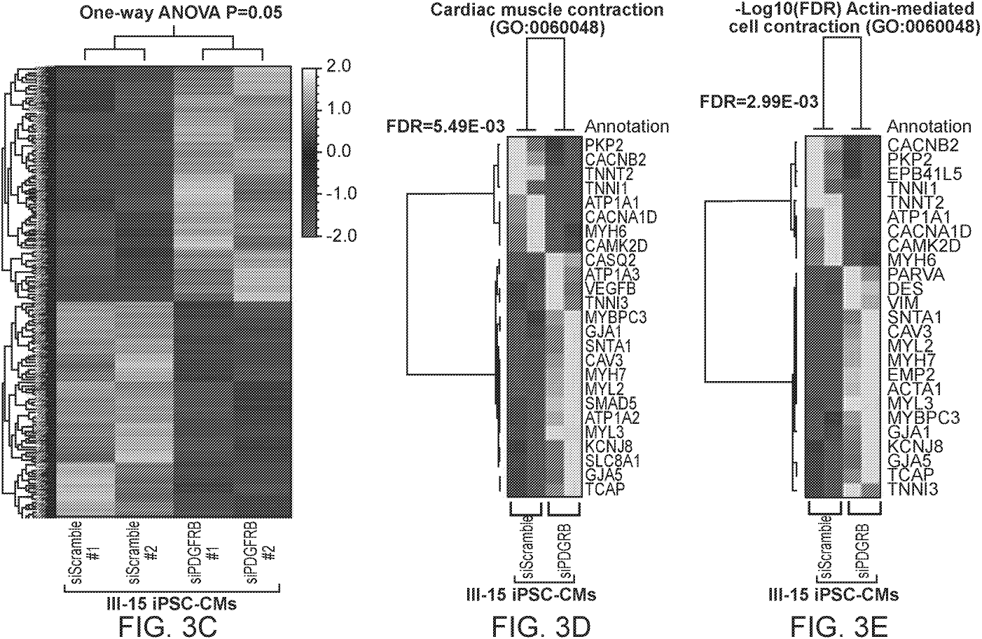

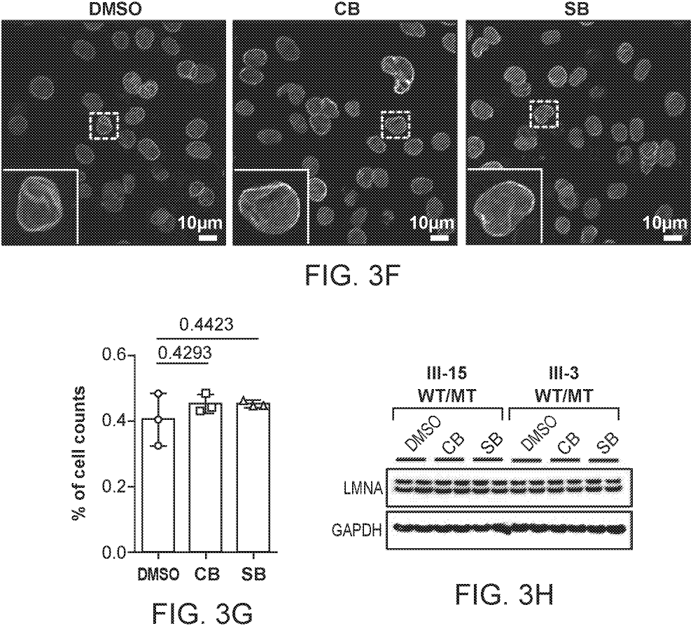

A total of 910 genes were identified that were differentially expressed between the treated and the untreated groups. GO term analysis of downregulated genes in the treated groups showed a high enrichment of genes related to heart functions, including muscle contraction, the regulation of cardiac conduction and ion transport. We confirmed significant changes in the expression of genes related to cardiac muscle contraction and actin-mediated cell contraction through the knockdown of PDGFRB in K117fs iPSC-CMs. We found that there were no differences in the lamin A/C level or the nuclear structure after treatment with crenolanib or sunitinib. Taken together, the data shown in FIGS. 3A-3H confirm that the lamin A/C haploinsufficiency causes the abnormal activation of the PDGF signaling pathway, leading to the development of arrhythmias in LMNA-related DCM.

These results confirm the ability of the present invention to prevent systolic dysfunction in LMNA-mutated cardiomyocytes. Combined with the above restoration of normal calcium signaling, the methods disclosed herein prevented the arrythmia and systolic dysfunction associated with LMNA-mutated dilated cardiomyopathy. Thus, the present invention effectively prevents the development of systolic dysfunction and treats the arrhythmic phenotype associated with LMNA-mutated dilated cardiomyopathy.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word "a" or "an" when used in conjunction with the term "comprising" in the claims and/or the specification may mean "one," but it is also consistent with the meaning of "one or more," "at least one," and "one or more than one." The use of the term "or" in the claims is used to mean "and/or" unless explicitly indicated to refer to alternatives only or the alternatives are mutually exclusive, although the disclosure supports a definition that refers to only alternatives and "and/or." Throughout this application, the term "about" is used to indicate that a value includes the inherent variation of error for the device, the method being employed to determine the value, or the variation that exists among the study subjects.

As used in this specification and claim(s), the words "comprising" (and any form of comprising, such as "comprise" and "comprises"), "having" (and any form of having, such as "have" and "has"), "including" (and any form of including, such as "includes" and "include") or "containing" (and any form of containing, such as "contains" and "contain") are inclusive or open-ended and do not exclude additional, unrecited features, elements, components, groups, integers, and/or steps, but do not exclude the presence of other unstated features, elements, components, groups, integers and/or steps. In embodiments of any of the compositions and methods provided herein, "comprising" may be replaced with "consisting essentially of" or "consisting of". As used herein, the term "consisting" is used to indicate the presence of the recited integer (e.g., a feature, an element, a characteristic, a property, a method/process step or a limitation) or group of integers (e.g., feature(s), element(s), characteristic(s), property(ies), method/process steps or limitation(s)) only. As used herein, the phrase "consisting essentially of" requires the specified features, elements, components, groups, integers, and/or steps, but do not exclude the presence of other unstated features, elements, components, groups, integers and/or steps as well as those that do not materially affect the basic and novel characteristic(s) and/or function of the claimed invention.

The term "or combinations thereof" as used herein refers to all permutations and combinations of the listed items preceding the term. For example, "A, B, C, or combinations thereof" is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, "about", "substantial" or "substantially" refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skill in the art recognize the modified feature as still having the required characteristics and capabilities of the unmodified feature. In general, but subject to the preceding discussion, a numerical value herein that is modified by a word of approximation such as "about" may vary from the stated value by at least ±1, 2, 3, 4, 5, 6, 7, 10, 12 or 15%.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words "means for" or "step for" are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

Anderson, P. O., Knoben, J. E., & Troutman, W. G. (2002). Handbook of clinicaldrug data (10th ed.). New York ; Toronto: McGraw-Hill Medical Pub. Division.

Brayson, D., & Shanahan, C. M. (2017). Current insights into LMNA cardiomyopathies: Existing models and missing LlNCs. Nucleus, 8(1), 17-33. doi:10.1080/19491034.2016.1260798

Chintalgattu, V., Ai, D., Langley, R. R., Zhang, J., Bankson, J. A., Shih, T. L., ... Khakoo, A. Y. (2010). Cardiomyocyte PDGFR-beta signaling is an essential component of the mouse cardiac response to load-induced stress. J Clin Invest, 120(2), 472-484. doi:10.1172/JCI39434

Diecke, S., Lu, J., Lee, J., Termglinchan, V., Kooreman, N. G., Burridge, P. W., ... Wu, J. C. (2015). Novel codon-optimized mini-intronic plasmid for efficient, inexpensive, and xeno-free induction of pluripotency. Sci Rep, 5, 8081. doi:10.1038/srep08081

Goidescu, C. M. (2013). Dilated cardiomyopathy produced by lamin A/C gene mutations. ClujulMed, 86(4), 309-312.

Goldstein, A., Pratt, W. B., & Taylor, P. (1990). Principles of drug action : the basis of pharmacology (3rd ed.). New York: Churchill Livingstone.

Goodman, L. S., Hardman, J. G., Limbird, L. E., & Gilman, A. G. (2001). Goodman and Gilman's the pharmacological basis of therapeutics (10th ed.). New York: McGraw-Hill.

Greensmith, D. J. (2014). Ca analysis: an Excel based program for the analysis of intracellular calcium transients including multiple, simultaneous regression analysis. Comput Methods Programs Biomed, 113(1), 241-250. doi:10.1016/j.cmpb.2013.09.004

Hershberger, R. E., Lindenfeld, J., Mestroni, L., Seidman, C. E., Taylor, M. R., Towbin, J. A., & Heart Failure Society of, A. (2009). Genetic evaluation of cardiomyopathy-a Heart Failure Society of America practice guideline. J Card Fail, 15(2), 83-97. doi:10.1016/j.cardfail.2009.01.006

Hershberger, R. E., & Morales, A. (2016). LMNA-Related Dilated Cardioimyopatliy. In M. P. Adam, H. H. Ardinger, R. A. Pagon, S. E. Wallace, L. J. H. Bean, K. Stephens, & A. Amemiya (Eds.), GeneReviews((R)). Seattle (WA).

Hershberger, R. E., Morales, A., & Siegfried, J. D. (2010). Clinical and genetic issues in dilated cardiomyopathy: a review for genetics professionals. Genet Med, 12(11), 655-667. doi:10.1097/GIM.0b013e3181f2481f

Katzung, B. G. (2004). Basic & clinical pharmacology (9th ed.). New York: Lange Medical Books/McGraw Hill.

Kodo, K., Ong, S. G., Jahanbani, F., Termglinchan. V., Hirono, K., InanlooRahatloo, K., ... Wu, J. C.

. iPSC-derived cardiomyocytes reveal abnormal TGF-beta signaling in left ventricular non-compaction cardiomyopathy. Nat Cell Biol, 18(10), 1031-1042. doi:10.1038/ncb3411

Lam, C. K., Zhao, W., Cai, W., Vafiadaki, E., Florea, S. M., Ren, X., ... Kranias, E. G. (2013). Novel role of HAX-1 in ischemic injury protection involvement of heat shock protein 90. Circ Res, 112(1), 79-89. doi:10.1 161/CIRCRESAHA.1 12.279935

Lee,J., Shao, N.Y., Paik, D.T.,Wu, H., Guo, H.,Termglinchan, V., ...Wu, J. C. (2018). SETD7 Drives Cardiac Lineage Commitment through Stage-Specific Transcriptional Activation. Cell Stem Cell, 22(3), 428-444 e425. doi:10.1016/j.stem.2018.02.005

Lee, J., Termglinchan, V., Diecke, S., Itzhaki, I., Lam, C. K., Garg, P., ... Wu, J. C. (2019). Activation of PDGF pathway links LMNA mutation to dilated cardiomyopathy. Nature, 572(7769), 335-340. doi:10.1038/s41586-019-1406-x

Lin, F., & Worman, H. J. (1993). Structural organization of the human gene encoding nuclear, lamin A and nuclear lamin C. JBiol Chem, 268(22), 16321-16326.

Lu, J. T., Muchir, A., Nagy, P. L., & Worman, H. J. (2011). LMNA cardiomyopathy: cell biology and genetics meet clinical medicine. Dis Model Mech, 4(5), 562-568. doi:10.1242/dmm.006346

Martindale, W., Reynolds, J. E. F., & Royal Pharmaceutical Society of Great Britain. Council. (1996). The extra pharmacopoeia (31st ed.). London: Royal Pharmaceutical Society.

Mattout, A., Cabianca, D. S., & Gasser, S. M. (2015). Chromatin states and nuclear organization in development--a view from the nuclear lamina. Genome Biol, 16, 174. doi: 10.1186/s13059-015-0747-5 Perovanovic, J., Dell'Orso, S., Gnochi, V. F., Jaiswal, J. K., Sartorelli, V., Vigouroux, C., ... Hoffman, E. P. (2016). Laminopathies disrupt epigenomic developmental programs and cell fate. Sci TranslMed, 8(335), 335ra358. doi:10.1126/scitranslmed.aad4991

Remington, J. P., & Gennaro, A. R. (2000). Remington : the science and practice of pharmacy (20th ed ed.). Baltimore, Md.: Lippincott Williams & Wilkins.

Richardson, P., McKenna, W., Bristow, M., Maisch, B., Mautner, B., O'Connell, J.,... Nordet, P. (1996). Report of the 1995 World Health Organization/!nternational Society and Federation of Cardiology Task Force on the Definition and Classification of cardiomyopathies. Circulation, 93(5), 841-842. doi:10.1161/01.cir.93.5.841

Solovei, I., Wang, A. S., Thanisch, K., Schmidt, C. S., Krebs, S., Zwerger, M., ... Joffe, B. (2013). LBR and lamin A/C sequentially tetherperipheral heterochromatin and inversely regulate differentiation. Cell, 152(3), 584-598. doi:10.1016/j.cell.2013.01.009

WHO/ISFC. (1980). Report of the WHO/ISFC task force on the definition and classification of cardiomyopathies. Br Head J, 44(6), 672-673. doi:10.1136/hrt.44.6.672

Wilkes, G. M. (2016). Oncology Nursing Drug Handbook 2016 (20 ed.). Sudbury: Jones & Bartlett Publishers.

1. A method for treating and/or preventing dilated cardiomyopathy due to an LMNA mutation, the method comprising:
administering to a subject a therapeutically effective amount of an inhibitor of PDGF signaling.

2. The method of claim 1, wherein the inhibitor of PDGF signaling is crenolanib or salt thereof.

3. The method of claim 1, wherein the LMNA mutation results in lamin A/C haploinsufficiency and abnormal activation of the PDGFR signaling pathway.

4. The method of claim 1, wherein LMNA-mediated hyperactivation of PDGFRβ signaling pathways in cardiomyocytes leads to changes in gene and protein expression to exhibit a proarrhythmic phenotype.

5. The method of claim 1, wherein the inhibitor of PDGF signaling prevents or reduces LMNA-mediated hyperactivation of PDGFRβ in cardiomyocytes.

6. The method of claim 1, wherein the inhibitor of PDGF signaling thereof reduces the level of phosphorylation of CAMK2D and RYR2.

7. The method of claim 1, wherein the inhibitor of PDGF signaling prevents or reduces the pro-arrhythmic phenotype of cardiomyocytes carrying an LMNA mutation.

8. The method of claim 1, wherein the inhibitor of PDGF signaling downregulates genes associated with muscle contraction and regulation of cardiac conduction.

9. The method of claim 1, wherein the inhibitor of PDGF signaling prevents and/or treats systolic and/or diastolic dysfunction associated with LMNA mutation.

10. The method of claim 2, wherein the therapeutically effective amount of crenolanib is from about 50 mg to 500 mg per day, 100 to 450 mg per day, 200 to 400 mg per day, 300 to 500 mg per day, 350 to 500 mg per day, or 400 to 500 mg per day.

11. The method of claim 1, wherein the inhibitor of PDGF signaling is administered at least one of continuously, intermittently, systemically, or locally.

12. The method of claim 1, wherein the inhibitor of PDGF signaling is administered orally, intravenously, or intraperitoneally.

13. The method of claim 1, wherein the inhibitor of PDGF signaling is administered up to three times a day for as long as the subject is in need of a treatment for cardiovascular disease.

14. The method of claim 2, wherein the crenolanib is one or more of crenolanib besylate, crenolanib phosphate, crenolanib lactate, crenolanib hydrochloride, crenolanib citrate, crenolanib acetate, crenolanib toluenesulphonate, and crenolanib succinate.

15. The method of claim 1, wherein the inhibitor of PDGF signaling is Imatinib; Sunitinib; Sorafenib; Pazopanib; Nilotinib; Cediranib; Motesanib; Axitinib; Linifenib; Dasatinib; Quizartinib; or Ponatinib.
